(19) United States
(12) Patent Application Publication
(10) Pub. No.: US 2009/0123504 A1
(43) Pub. Date: May 14, 2009

(54) OLIVE OIL FORMULATION FOR PAIN RELIEF

(75) Inventors: Joseph R. Feldkamp, Appleton, WI (US); Laura Serra, Greenville, WI (US); Pierre Joseph, Hamilton, NJ (US); Corey Cunningham, Appleton, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: KIMBERLY-CLARK WORLDWIDE, INC., Neenah, WI (US)

(21) Appl. No.: 11/938,596

(22) Filed: Nov. 12, 2007

(51) Int. Cl. A61K 9/00; A61K 31/22; A61P 29/00

(52) U.S. Cl. 424/402; 514/549

(57) ABSTRACT

The present disclosure generally relates to methods and compositions for reducing inflammation and/or pain, and more particularly, to compositions comprising an NSAID and oleocanthal and/or a related compound. The compositions are particularly suitable for topical delivery and may be used to relieve the effects of arthritis.

### BACKGROUND OF THE DISCLOSURE

The present disclosure generally relates to methods and compositions for reducing inflammation and/or pain, and more particularly, to compositions comprising an NSAID and oleocanthal and/or a related compound. The compositions are particularly suitable for topical delivery and may be used to relieve the effects of arthritis.

Topical routes of drug administration are often desirable, because problems associated with other drug delivery means can be avoided. For instance, oral administration of drugs is often associated with variable absorption and metabolism of the drug and gastrointestinal irritation. In particular, the gastrointestinal irritation associated with the oral administration of non-steroidal anti-inflammatory drugs (NSAIDs), such as aspirin, ibuprofen, and naproxen, among others, is well documented. Oral administration of these and other NSAIDs can cause substantial irritation of the gastrointestinal tract, including gastrointestinal ulceration and bleeding, nausea, and dyspepsia. Oral NSAID administration may also result in other systemic adverse effects, such as headache, dizziness, salt and fluid retention, and hypertension. Furthermore, a significant amount of the drug may be lost as a result of oral administration, due to partial metabolism of the drug by the liver prior to reaching the bloodstream.

In an effort to overcome the drawbacks of oral administration of NSAIDs, an ibuprofen semi-solid formulation was recently introduced that can be applied directly to skin above the source of inflammation. Although such a semi-solid may avoid some of the traditional problems associated with administration of NSAIDs (e.g., gastrointestinal irritation), there are other problems associated with topical administration of these drugs.

For instance, the skin provides a protective barrier against foreign materials and infections and helps protect the body from water loss. The layer of skin primarily responsible for this barrier function is the epidermis, and more particularly, the uppermost layer of the epidermis (i.e., the stratum corneum or horny layer). In mammals, the stratum corneum consists of horny skin cells (i.e., corneocytes) embedded in a lipid matrix. This structure is often referred to as a “brick and mortar” model, with the corneocytes being the “bricks” and the epidermal lipids being the “mortar.” The structure of the stratum corneum provides a strong barrier to the diffusion of molecules through the skin, often restricting diffusion through the skin to molecules having a certain size, solubility, and/or lipophilicity. This presents a problem for the diffusion of certain NSAIDs, such as ibuprofen, which, under certain conditions, have a relatively low solubility in fats and oils, and may precipitate and form an insoluble salt.

There is thus a need for compositions useful for the relief of inflammation that may be effectively delivered topically.

### SUMMARY OF THE DISCLOSURE

The present disclosure generally relates to methods and compositions for reducing inflammation and/or pain, and more particularly, to compositions comprising an NSAID and oleocanthal and/or a related compound. The compositions are particularly suitable for topical delivery and may be used to relieve the effects of arthritis.

In one aspect, the present disclosure is directed to a composition for relief of inflammation or pain, the composition comprising a pharmaceutically acceptable carrier, at least about 0.04% (by weight of the composition) of a first active ingredient, and an effective amount of a second active ingredient, wherein the first active ingredient has the general structure:

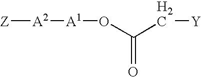

wherein Z is

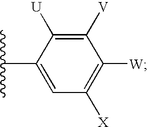

u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH(OCH₃); A¹ is selected from the group consisting of CH₂, CHCH₃, CH(CH₂CH₃), and C(CH₃)₂, CH(NH₂); and Y is selected from the group consisting of

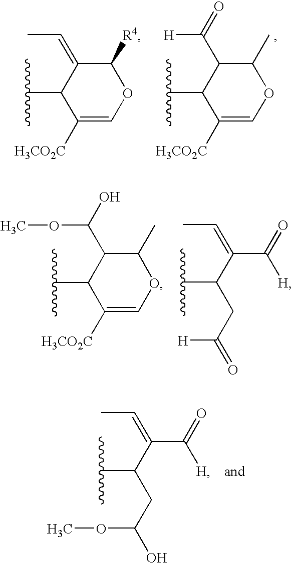

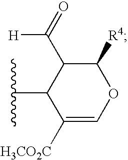

wherein R⁴ is selected from the group consisting of —H and

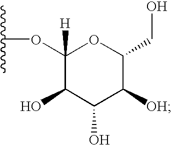

and wherein the second active ingredient is a non-steroidal anti-inflammatory drug.

In another aspect, the present disclosure is directed to a composition for relief of pain or inflammation, the composition comprising a pharmaceutically acceptable carrier, a first active ingredient, a second active ingredient, and a skin penetration enhancer, wherein the first active ingredient is as set forth herein, and the second active ingredient is a non-steroidal anti-inflammatory drug.

In another aspect, the present disclosure is directed to a method for relieving pain or inflammation in a body portion. The method comprises applying a composition to the body portion, the composition comprising a pharmaceutically acceptable carrier, at least about 0.04% (by weight of the composition) of a first active ingredient, and a second active ingredient; and covering the body portion with a barrier substrate; wherein the second active ingredient is a non-steroidal anti-inflammatory drug and the first active ingredient is as set forth herein.

In yet another aspect, the present disclosure is directed to a method for relieving pain or inflammation in a body portion. The method comprises covering the body portion with a barrier substrate, the barrier substrate having an interior surface for contacting the body portion; wherein the interior surface of the barrier substrate comprises a composition, the composition comprising a pharmaceutically acceptable carrier, at least about 0.04% (by weight of the composition) of a first active ingredient, and a second active ingredient, wherein the second active ingredient is a non-steroidal anti-inflammatory drug, and the first active ingredient is as set forth herein.

In still another aspect, the present disclosure is directed to a system for relieving pain or inflammation in a body portion. The system comprises a barrier substrate comprising an interior surface for contacting the body portion and an exterior surface opposite the interior surface; and a composition comprising a pharmaceutically acceptable carrier, at least about 0.04% (by weight of the composition) of a first active ingredient, and a second active ingredient, wherein the second active ingredient is a non-steroidal anti-inflammatory drug, and the first active ingredient is as set forth herein.

Other objects and features will be in part apparent and in part pointed out hereinafter.

### DETAILED DESCRIPTION OF THE DISCLOSURE

In accordance with the present disclosure, it has been discovered that non-steroidal anti-inflammatory drugs (NSAIDs) in combination with the anti-inflammatory compound oleocanthal or related compounds, can be delivered topically for relief of inflammation and/or pain. More particularly, NSAIDs and oleocanthal or related compounds can be introduced into various compositions, such as gels, lotions, suspensions, creams, and sprays, which may be topically applied at the source of inflammation and/or pain. Advantageously, the NSAID/oleocanthal-containing compositions overcome several of the drawbacks associated with traditional topical formulations containing only NSAIDs as active ingredients.

NSAIDs are commonly used to treat inflammation and pain, and may be of particular use in treating the symptoms associated with arthritis. However, as noted above, oral administration of these medications may lead to gastrointestinal tract irritation. Furthermore, it has been difficult to administer ibuprofen and other similar NSAIDs such that they will permeate the stratum corneum. Consequently, formulating a NSAID-containing composition that is capable of penetrating through the stratum corneum in effective amounts is desired.

It has now been discovered that topical compositions comprising NSAIDs may be formulated such that the NSAID present in the composition will readily penetrate through skin. The compositions of the present disclosure are thus suitable for topical administration of NSAIDs to treat pain and/or inflammation, such as is commonly associated with arthritis and other conditions.

More particularly, it has been discovered that maintaining an NSAID in its non-ionized form in a composition will improve the ability of the NSAID to penetrate through skin. As noted above, the external skin barrier is lipophilic. While non-ionized forms of NSAIDs are soluble in oils and lipids, and may thus penetrate through the lipophilic external skin barrier, ionized NSAIDs are insoluble in oils and lipids. Consequently, ionized NSAIDs will be unable to penetrate through the external skin barrier.

The topical compositions of the present disclosure are advantageously formulated such that at least about 50%, more preferably at least about 75%, and more preferably at least about 90% of the NSAID present in the aqueous portion of the composition is in non-ionized form.

More particularly, most NSAIDs will ionize in aqueous carriers such as water, but will remain in their non-ionized form, and thus suitable for transdermal delivery, in the presence of non-aqueous carriers, such as oils. The degree of ionization of the NSAID in the aqueous phase of a composition will depend on the pH of the composition and the specific pKa of the NSAID. For instance, the NSAIDs ibuprofen, naproxen, and acetylsalicylic acid (i.e., aspirin) will typically ionize if the pH of the water phase is above 4.5. Once ionized, the NSAID will not be soluble in the oil phase of the composition or will precipitate out of the composition, and thus will be ineffective at penetrating through the external skin barrier.

It is thus desirable to maintain the NSAID in its non-ionized form in the composition. This may be achieved by reducing, or entirely eliminating, aqueous components from the composition. Previously known NSAID formulations were typically creams or other oil in water emulsions that contained a substantial fraction of the NSAID in the water phase in its ionized form. Consequently, a substantial portion of the NSAIDs in these compositions was unable to effectively penetrate through the lipophilic external skin barrier, and an important fraction of the NSAID dose was lost.

In contrast, the compositions of the present disclosure may comprise, in one embodiment, limited amounts of aqueous components. Preferably, the compositions of the present disclosure will comprise less than about 10% (by weight of the composition) of water, more preferably less than about 5% (by weight of the composition) of water, and more preferably will be non-aqueous (i.e., will comprise 0% (by weight of the composition) of water).

It should be understood that in certain embodiments, the compositions of the present disclosure may comprise limited amounts of water. For instance, the compositions may comprise up to about 10% (by weight of the composition) of water. As discussed above, the degree of ionization of the NSAID in the aqueous phase of the composition will depend on the pH of the aqueous phase and the specific pKa of the NSAID. In embodiments where the composition comprises water, it is generally preferable that the pH of the aqueous phase be such that at least about 50%, more preferably at least about 75%, and more preferably at least about 90% of the NSAID present in the aqueous phase is in non-ionized form.

Because the NSAIDs present in the composition of the present disclosure are substantially, if not entirely, in non-ionized form, they are readily absorbed through the skin when the composition is topically applied. The compositions of the present disclosure may thus be advantageously used for treatment of inflammation and/or pain, such as is associated with different types of arthritic disorders, injuries, and other disorders.

The compositions of the present disclosure may comprise any NSAID in non-ionized form. Examples of suitable NSAIDs include, for example, ibuprofen, aspirin, difunisal, etodolac, indometacin, nabumeton, sulindac, tolmetin, caprofen, fenbufen, flubiprofen, ketoprofen, ketorolac, loxoprofen, naproxen, oxaprozin, tiaprofenic acid, suprofen, mefenaminic acid, phenylbutazone, piroxicam, meloxicam, celecoxib, etoricoxib, lumiracoxib, parecoxib, rofecoxib, valdecoxib, nimesulide, and combinations thereof. Preferably, the NSAID is selected from the group consisting of the following classes of NSAID: profens, fenamic acids, arylalkanoic acids, salicylates, and combinations thereof.

The amount of NSAID present in the composition may vary depending on the ability of the individual NSAID to penetrate through the skin, the NSAID's potency, and its half-life, but will preferably be present in an amount effective to reduce inflammation and/or pain. Typically, the composition will comprise from about 0.02% (by weight of the composition) to about 10.0% (by weight of the composition) of NSAID,

In addition to an NSAID, the compositions of the present disclosure may further comprise oleocanthal, its analogues, and/or related compounds. Certain types of olive oil, and in particular extra virgin olive oil, have been shown to contain a naturally occurring compound that acts in a similar manner to traditional NSAIDs in the relief of inflammation and pain. The compound, called oleocanthal, is generally thought to be responsible for the stinging sensation produced in the throat when certain olive oils are ingested, and is believed to inhibit the activity of the cyclooxygenase-1 (COX-1) and cyclooxygenase-2 (COX-2) enzymes. Because the inhibition of COX-1 and COX-2 enzymes underlies the anti-inflammatory actions of ibuprofen and other NSAIDs, oleocanthal can advantageously be used in a manner similar to other NSAIDs to relieve inflammation and pain. Oleocanthal has the following chemical structure:

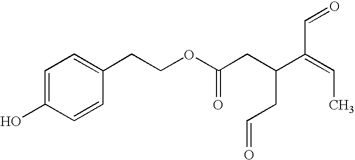

In accordance with the present disclosure, it has been discovered that oleocanthal and related compounds do not suffer from the same drawbacks as ibuprofen traditionally has when administered topically. More particularly, oleocanthal and related compounds are readily highly soluble in fats and oils, and remain in their neutral, non-ionized state over a broad pH range. Without wishing to be bound to any particular theory, it is believed that the ability of oleocanthal and related compounds to remain in a neutral state over a broad pH range contributes to the ability of oleocanthal and related compounds to more readily penetrate the stratum corneum, even in the presence of water, as compared to ibuprofen and similar NSAIDs. As noted above, NSAIDs will generally exhibit better penetration when in a neutral, or non-ionized, state. Since the phenolic group in the structure of oleocanthal generally does not begin to ionize until about pH 9.5, oleocanthal and related compounds will typically remain in their neutral, non-ionized form in compositions having a pH up to about 9.5. This is advantageous for topical delivery. Since most transdermal compositions are formulated to have a pH of from about 3.5 to about 8.5, and more typically from about 5.0 to about 6.0, oleocanthal and related compounds will mostly be in their neutral, non-ionized states when the transdermal composition is contacted with skin. This is true even if the composition comprises an aqueous phase. As noted above, ibuprofen and similar NSAIDs, which contain a carboxylic acid group that may begin to ionize at a pH of about 4.5, will be all or partly in their ionized state (and thus insoluble) when a transdermal composition containing an aqueous phase is contacted with skin. Consequently, oleocanthal and related compounds penetrate the stratum corneum more readily than ibuprofen and other similar NSAIDs.

The compositions of the present disclosure may thus advantageously comprise oleocanthal and/or related compounds in addition to NSAIDs. Because oleocanthal and related compounds can readily penetrate the external skin barrier, inclusion of oleocanthal and/or related compounds in the compositions of the present disclosure may help increase the effectiveness of the composition. Furthermore, non-ionized ibuprofen and similar NSAIDs are soluble in olive oil, which, as noted above, is a source of oleocanthal. Thus, compositions having good skin penetration of active ingredients may be formulated to comprise oleocanthal-containing olive oil and an NSAID.

As noted above, a variety of compounds structurally related to oleocanthal act in a manner similar to oleocanthal and may also be included in the compositions of the present disclosure. The oleocanthal-related compounds that may be used in the compositions of the present disclosure have the following general structure (hereinafter Structure 1):

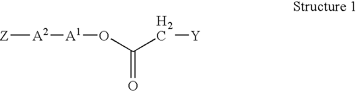

wherein Z is

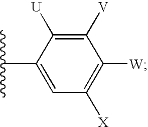

u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH(OCH₃); A¹ is selected from the group consisting of CH₂, CHCH₃, CH(CH₂CH₃), and C(CH₃)₂, CH(NH₂); and Y is selected from the group consisting of

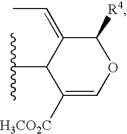

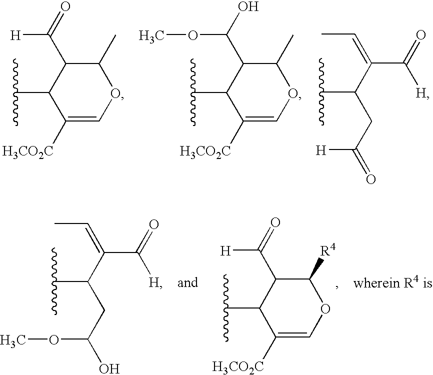

selected from the group consisting of —H and

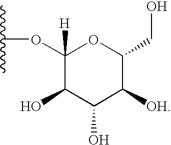

Preferably, the oleocanthal-related compound is selected from the group consisting of

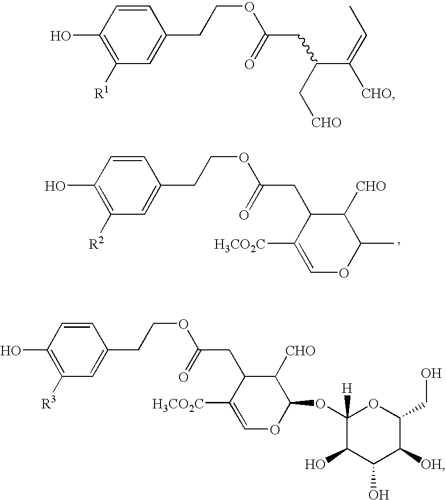

The oleocanthal used in the compositions described herein may be obtained from any suitable source. For instance, oleocanthal can be extracted from certain olive oils, such as extra virgin olive oil, using any suitable procedure known in the art, such as that described in Andrewes, et al., J. Agric. Food Chem., Vol. 51, No. 5, pp. 1415-1420 (2003). More specifically, in this procedure 200 g of olive oil is dissolved in 400 mL of hexane and extracted with 400 mL of a 60:40 ethanol:water mixture for 2 minutes. The aqueous ethanol/water phase is collected and washed with a second 400 mL volume of hexane. The hexane phase is discarded and the ethanol/water phase is rotary evaporated to dryness using a water bath at 40° C. The dried extract, which contains a number of olive oil components, may then be further separated using chromatographic means, such as high performance liquid chromatography (HPLC). Alternately, the oleocanthal and its analogues may be synthesized using any suitable procedure, such as that described in Smith, et al., Org. Lett., Vol. 7, No. 22, pp. 5075-5078 (2005), and Smith, III, et al., J. Org. Chem., Vol. 72, pp. 6891-6900 (2007).

In order to deliver effective amounts of oleocanthal, the compositions comprise at least about 0.04% (by weight of the composition) of oleocanthal. More preferably, the compositions comprise from about 0.04% (by weight of the composition) to about 10.0% (by weight of the composition) of oleocanthal, more preferably from about 0.10% (by weight of the composition) to about 5.0% (by weight of the composition) of oleocanthal, and still more preferably about 0.50% (by weight of the composition) of oleocanthal. Oleocanthal obtained from extraction and/or organic synthesis may be added directly to the composition and/or the oleocanthal present in the composition may be the result of the addition of oleocanthal-containing olive oil to the composition. In either event, the compositions will preferably comprise at least about 0.04% (by weight of the composition) of oleocanthal.

Thus, in one aspect, the present disclosure is directed to compositions for the relief of inflammation and/or pain, wherein the compositions comprise an effective amount of a first active ingredient and an effective amount of a second active ingredient, wherein the second active ingredient is an NSAID and the first active ingredient has general Structure 1. Advantageously, the compositions may be topically applied at the source of inflammation and/or pain. In particular, the compositions are useful for reducing skin, joint, and muscle inflammation, and for reducing the discomfort of arthritis.

In addition to an NSAID and oleocanthal and/or related compound, the compositions of the present disclosure may also include a skin penetration enhancer or mixture of skin penetration enhancers in order to increase the permeability of the oleocanthal and NSAID into the skin. More particularly, skin penetration enhancers help to improve transport of oleocanthal and NSAID across the stratum corneum by assisting in the penetration of the oleocanthal and NSAID through the skin's stratum corneum. This may be accomplished by a number of different mechanisms including, for example, by extracting lipids from the stratum corneum, increasing the partitioning of the NSAID and oleocanthal into the skin, and disrupting the lipid bilayer of the stratum corneum, thus rendering the stratum corneum structure more fluid and increasing the ability of the oleocanthal and NSAID to diffuse through the stratum corneum.

Examples of suitable skin penetration enhancers include sulfoxides, alcohols, fatty acids, fatty acid esters, polyols, amides, surfactants, terpenes, alkanones, and organic acids, among others.

Specific examples of suitable sulfoxides include dimethylsulfoxide (DMSO) and decylmethylsulfoxide, among others.

Suitable alcohols include alkanols such as ethanol, propanol, butanol, pentanol, hexanol, octanol, n-octanol, nonanol, decanol, 2-butanol, 2-pentanol, and benzyl alcohol; fatty alcohols, such as caprylic alcohol, decyl alcohol, lauryl alcohol, 2-lauryl alcohol, myristyl alcohol, cetyl alcohol, stearyl alcohol, oleyl alcohol, linoleyl alcohol, and linolenyl alcohol; and isopropyl alcohol.

Examples of suitable fatty acids include linear fatty acids such as valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic acid, and isostearic acid.

Examples of suitable fatty acid esters include aliphatic fatty acid esters such as isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide.

Examples of suitable polyols include propylene glycol, butylene glycol, polyethylene glycol, ethylene glycol, diethylene glycol, triethylene glycol, dipropylene glycol, ethoxydiglycol, pentylene glycol, glycerol, propanediol, butanediol, pentanediol, hexanetriol, and glycerin.

Examples of suitable amides include urea, dimethylacetamide, diethyltoluamide, dimethylformamide (DMF), dimethyloctamide, dimethyldecamide, biodegradable cyclic urea (e.g., 1-alkyl-4-imidazoline-2-one), pyrrolidone derivatives, biodegradable pyrrolidone derivatives (e.g., fatty acid esters of N-(2-hydroxyethyl)-2-pyrrolidone), cyclic amides, hexamethylenelauramide and its derivatives, diethanolamine, and triethanolamine. Examples of pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkylpyrrolidone, and N-methylpyrrolidone. Examples of cyclic amides include 1-dodecylazacycloheptane-2-one (e.g., Azone®), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyldodecyl)azacyclohaptane-2-one, 1-geranylazacyclohexane-2-one, 1-geranylazacyclopentan-2,5-dione, and 1-farnesylazacyclopentan-2-one.

Suitable surfactants may include anionic surfactants, cationic surfactants, nonionic surfactants, bile salts, and lecithin. Examples of suitable anionic surfactants include sodium laurate, sodium lauryl sulfate, and sodium laureth sulfate. Suitable cationic surfactants include cetyltrimethyl ammonium bromide, tetradecyltrimethylammonium bromide, benzalkonium chloride, octadecyltrimethylammonium chloride, cethylpyridinium chloride, dodecyltrimethylammonium chloride, and hexadecyultrimethylammonium chloride. Examples of suitable nonionic surfactants include poloxamer 231, poloxamer 182, poloxamer 184, Brij® 30 (polyoxyethylene (4) lauryl ether), Brij® 93 (polyoxyethylene (2) oleyl ether), Brij® 96 (polyoxyethylene (20) oleyl ether), Brij® 99 (polyoxyl (10) oleyl ether), Span® 20 (sorbitan monolaurate), Span® 40 (sorbitane monopalmitate), Span® 60 (sorbitane monostearate), Span® 80 (sorbitane monooleate), Span® 85 (sorbitane trioleate), TWEEN® 20 (polyethylene glycol sorbitan monolaurate; polyoxyethylene (20) sorbitan monolaurate), TWEEN® 40 (polyoxyethylene (20) sorbitan monopalmitate), TWEEN® 60 (polyethylene glycol sorbitan monostearate; polyoxyethylene (20) sorbitan monostearate), TWEEN® 80 (polyethylene glycol sorbitan monooleate; polyoxyethylene (20) sorbitan monooleate), Myrj® 45 (polyoxyethylene (8) stearate), Myrj® 51 (polyoxyethylene stearate), Myrj® 52 (polyoxyethylene stearate), and Miglyol 840 (propylene glycol dicaprylate/dicaprat), among others. Examples of suitable bile salts include sodium cholate, and sodium salts of taurocholic, glycholic, and desoxycholic acids.

Suitable terpenes include hydrocarbons (e.g., D-limonene, α-pinene, β-carene, etc.), alcohols (e.g. α-terpineol, terpinen-4-ol, carvol, etc.), ketones (e.g., carvone, pulegone, piperitone, menthone, etc.), oxides (e.g., cyclohexene oxide, limonene oxide, α-pinene oxide, cyclopentene oxide, 1,8-cineole, etc.), and oils (e.g., ylang ylang, anise, chenopodium, eucalyptus, peppermint, etc.).

Examples of suitable alkanones include N-heptane, N-octane, N-nonane, N-decane, N-undecane, N-dodecane, N-tridecane, N-tetradecane, and N-hexadecane, among others.

Examples of suitable organic acids include salicylic acid and salicylates (including their methyl, ethyl, and propyl glycol derivatives), citric acid, and succinic acid, among others.

Other examples of suitable skin penetration enhancers are known in the art and include, for example, monoglycerides, polyglycosylated glycerides, glyceryl monoethyl ether, polysorbates, beta-cyclodextrin, cyclopentadecalactone, alkyl-2-(N,N-disubstituted amino)-alkanoate ester, 2-(n-nonyl)-1,3-dioxolane, isopropyl myristate, terpinol, menthol, cineol, monoolein, sodium oleate, oleyl oleate, laurylcapram, bisabolol, capaicin, and capsicum. Other examples of suitable skin penetration enhancers and a description of their mechanism of action may be found in Goodman and Barry, “Percutaneous Absorption,” in Mechanisms-Methodology-Drug Delivery, 2nd Edition, Bronaugh and Maibach, eds., 1989, pp. 567-593, Marcel Dekker, Inc., NY.

Preferably, the skin penetration enhancer is selected from the group consisting of n-octanol, D-limonene, oleic acid, cineol, isopropyl myristate, monooleate, monoolein, sodium oleate, oleyl oleate, laurylcapram, sodium lauryl sulfate, bisabolol, DMSO, ethanol, propanol, benzyl alcohol, lauryl alcohol, lauric acid, myristic acid, isopropyl palmitate, diisopropyl adipate, dimethyl isosorbide, propylene glycol, butylene glycol, polyethylene glycol, dipropylene glycol, ethoxydiglycol, pentylene glycol, urea, lecithin, sodium laureth sulfate, benzalkonium chloride, poloxamer 231, Brij® 30, Span® 20, Tween® 20, oil (e.g., ylang ylang, eucalyptus, peppermint), salicylic acid, citric acid, menthol, capaicin, capsicum, and combinations thereof. More preferably the skin penetration enhancer is selected from the group consisting of oleic acid, laurocapram, sodium lauryl sulphate, bisabolol, DMSO, ethanol, lauric acid, myristic acid, isopropyl myristate, isopropyl palmitate, diisopropyl adipate, dimethyl isosorbide, propylene glycol, butylene glycol, polyethylene glycol, dipropylene glycol, ethoxydiglycol, pentylene glycol, lecithin, benzalkonium chloride, D-limonone, oil (e.g., ylang ylang, eucalyptus, peppermint), salicylic acid, menthol, capaicin, capsicum, and combinations thereof.

Typically, the compositions of the present disclosure comprise from about 0.01% (by weight of the composition) to about 25% (by weight of the composition) of a skin penetration enhancer, more preferably from about 0.1% (by weight of the composition) to about 15% (by weight of the composition) of a skin penetration enhancer.

The compositions described herein may be employed with one or more conventional pharmaceutically-acceptable and compatible carrier materials useful for the desired application. The carrier can be capable of co-dissolving or suspending the oleocanthal and NSAID. Carrier materials suitable for use in the instant disclosure include those well-known for use in the cosmetic and medical arts as a basis for ointments, lotions, creams, salves, aerosols, gels, suspensions, sprays, foams, and the like, and may be used in their art-established levels.

Non-limiting examples of suitable carrier materials include water, emollients, sterols or sterol derivatives, natural and synthetic fats or oils, solidifying agents, viscosity enhancers, rheology enhancers, polyols, surfactants, alcohols, esters, silicones, clays, starch, cellulose, and other pharmaceutically acceptable carrier materials. As will be recognized by one skilled in the art, the relative amounts of components in the compositions of the disclosure that can be used to formulate the composition will be dictated by the nature of the composition. The levels can be determined by routine experimentation in view of the disclosure provided herein.

Thus, in one embodiment, the composition of the disclosure can optionally include one or more emollient, which typically acts to soften, soothe, and otherwise lubricate and/or moisturize the skin. Suitable emollients that can be incorporated into the compositions include oils such as petrolatum based oils, petrolatum, vegetable based oils, mineral oils, natural or synthetic oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof. The esters can be selected from cetyl palmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof can also suitably be used as emollients.

The composition may desirably include one or more emollient in an amount of from about 0.1% to about 95% by weight, more desirably from about 5% to about 75% by weight, and even more desirably from about 10% to about 50% by weight of the composition.

Stearol and stearol derivatives which are suitable for use in the compositions of the present disclosure include, but are not limited to cholestol, sitosterol, stigmasterol, ergosterol, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyidecanoate, ergocalciferol, tall oil sterol, soy sterol acetate, lanasterol, soy sterol, avocado sterols, fatty alcohols, and combinations thereof.

The composition of the invention can desirably include sterols, sterol derivatives or mixtures of both sterols and sterol derivatives in an amount of from about 0.1% to about 10% by weight, more desirably from about 0.5% to about 5% by weight, and even more desirably from about 0.8t to about 1% by weight of the composition.

The compositions of the disclosure can also include natural fats and oils. As used herein, the term “natural fat or oil” is intended to include fats, oils, essential oils, essential fatty acids, non-essential fatty acids, phospholipids, and combinations thereof. These natural fats and oils can provide a source of essential and non-essential fatty acids to those found in the skin's natural barrier. Suitable natural fats or oils can include citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, soybean oil, and combinations thereof.

The composition of the invention may desirably include fats and oils in an amount of from about 0.1% to about 95% by weight, more typically from about 5% to about 75% by weight, and even more typically from about 10% to about 50% by weight of the composition.

Preferably, the carrier comprises olive oil which, as discussed above, may be a natural source of oleocanthal. In one particular embodiment, the carrier is 100% olive oil, and is present in the composition in an amount of from about 10% (by weight of the composition) to about 90% (by weight of the composition).

Optionally, the composition may comprise a solidifying agent, which may function to solidify the composition so that the composition is a solid at room temperature, and may affect the hardness and melting point of the composition. The solidifying agent also provides a tackiness to the composition that improves the transfer to the skin of the wearer, such as when the composition is incorporated into a personal care product. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, and/or moisturizer. The solidifying agents may include waxes as well as compounds that perform functionally as waxes.

The solidifying agents can be selected from alkyl siloxanes, polymers, hydrogenated vegetable oils having a melting point of 35° C. or greater and fatty acid esters with a melting point of 35° C. or greater. Additionally, the solidifying agents can be selected from animal, vegetable and mineral waxes and alkyl silicones. Examples of solidifying agents include, but are not limited to, alkyl trimethylsilanes, beeswax, C₂₄-C₂₈ alkyl dimethicone, C₃₀ alkyl dimethicone, cetyl methicone, stearyl methicone, cetyl dimethicone, stearyl dimethicone, cerotyl dimethicone, candelilla wax, carnauba, cerasin, hydrogenated microcrystalline wax, jojoba wax, microcrystalline wax, lanolin wax, ozokerite, paraffin, spermaceti wax, cetyl esters, behenyl behenate, C₂₀-C₄₀ alkyl behenate, C₁₂-Cl₁₅ lactate, cetyl palmitate, stearyl palmitate, isosteryl behenate, lauryl behenate, stearyl benzoate, behenyl isostearate, cetyl myristate, cetyl octanote, cetyl oleate, cetyl ricinoleate, cetyl stearate, decyl oleate, diC₁₂-C₁₅ alkyl fumerate, dibehenyl fumerate, myristyl lactate, myristyl lignocerate, myristyl myristate, myristyl stearate, lauryl stearate, octyldodecyl stearate, octyldodecyl stearoyl stearate, olelyl arachidate, oleyl stearate, tridecyl behenate, tridecyl stearate, tridecyl stearoyl stearate, pentaerythrityl tetrabehenate, pentaerythrityl hydrogenated rosinate, pentaerythrityl distearate, pentaerythrityl tetraabeite, pentaerythrityl tetracocoate, pentaerythrityl tetraperlargonate, pentaerythrityl tetrastearate, theylene vinyl acetate, polyethylene, hydrogenated vegetable oil, hydrogenated squalene, squalene, hydrogenated coconut oil, hydrogenated jojoba oil, hydrogenated palm oil, hydrogenated palm kernel oil, hydrogenated olive oil, polyamides, metal stearates and other metal soaps, C₃₀-C₆₀ fatty alcohols, C₂₀₊ fatty acids, polypropylene, polystyrene, polybutane, polybutylene terephthalate, polydipentane, zinc stearate, and combinations thereof.

The composition may desirably include one or more solidifying agents in an amount of from about 0.1% to about 95% by weight, more desirably from about 5% to about 75% by weight, and even more desirably from about 10% to about 50% by weight of the composition.

Optionally, one or more viscosity enhancers may be added to the composition to increase the viscosity, to help stabilize the composition, such as when the composition is incorporated into a personal care product, thereby reducing migration of the composition and improve transfer to the skin. Suitable viscosity enhancers include polyolefin resins, lipophilic/oil thickeners, ethylene/vinyl acetate copolymers, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, clays, carbomers, acrylic based thickeners, and combinations thereof.

The composition may desirably include one or more viscosity enhancers in an amount of from about 0.1% to about 25% by weight, more desirably from about 0.5% to about 20% by weight, and even more desirably from about 1% to about 10% by weight of the composition.

The compositions of the disclosure may optionally further comprise rheology enhancers. Rheology enhancers may help increase the melt point viscosity of the composition so that the composition readily remains on the surface of a personal care product and does not substantially migrate into the interior of the product, while substantially not affecting the transfer of the composition to the skin. Additionally, the rheology enhancers help the composition to maintain a high viscosity at elevated temperatures, such as those encountered during storage and transportation.

Suitable rheology enhancers include combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, and styrene-ethylene/propylene copolymers and combinations thereof. Specifically, rheology enhancers such as mineral oil and ethylene/propylene/styrene copolymers, and mineral oil and butylene/ethylene/styrene copolymers (Versagel blends from Penreco) are particularly preferred. Also, Vistanex (Exxon) and Presperse (Amoco) polymers are particularly suitable rheology enhancers.

The composition of the invention can suitably include one or more rheology enhancer in an amount of from about 0.5% to about 5% percent by weight of the composition.

Examples of suitable polyols, surfactants, and alcohols include those listed above as skin penetration enhancers.

As noted above, in certain embodiments, the compositions may optionally comprise water. In these embodiments, the compositions may comprise up to about 10% (by weight of the composition) of water. As discussed above, the degree of ionization of the NSAID in the aqueous phase of the composition will depend on the pH of the composition and the specific pKa of the NSAID. In embodiments where the composition comprises water, it is therefore generally preferable that the pH of the aqueous phase of the composition be such that at least about 50%, more preferably at least about 75%, and more preferably at least about 90% of the NSAID present in the aqueous phase of the composition is in non-ionized form.

Optionally, the NSAID and oleocanthal-containing compositions may be formulated with a polar co-solvent to further increase the permeability of the NSAID and oleocanthal into the skin. Preferably, the polar co-solvent is fully miscible in the composition, and has a high affinity for the intercellular spaces in the stratum corneum. Without wishing to be bound by any particular theory, it is believed that polar co-solvents with such characteristics are driven by osmosis into the intercellular spaces in the stratum corneum, causing the stratum corneum to swell. In such a swollen state, the intercellular spaces are more liquid-like and disordered, which enables the NSAID and oleocanthal to more easily diffuse through the stratum corneum. Examples of suitable polar co-solvents for inclusion in the compositions of the present disclosure include, for example, ethanol, propylene glycol, butanol, isopropanol, propanol, isopropyl myristate, isopropyl palmitate, diisopropyl adipate, dimethyl isosorbide, butylene glycol, polyethylene glycol, dipropylene glycol, ethoxydiglycol, pentylene glycol, and combinations thereof. Preferably, the compositions of the present disclosure comprise from about 1% (by weight of the composition) to about 99% (by weight of the composition) of a polar co-solvent.

The compositions of the present disclosure may optionally further comprise ingredients to relieve irritation, such as anti-itch agents. The anti-itch agents may be present in the composition in an amount of from about 0.1% (by weight of the composition) to about 33% (by weight of the composition), more typically, from about 0.5% (by weight of the composition) to about 5% (by weight of the composition). Examples of suitable anti-itch agents are listed below, as well as the preferred concentration for each agent, given in percent by weight of the composition: lauromacrogols, benzocaine (about 5% to about 20%), butamben picrate (about 1%), dibucaine (about 0.25% to about 1%), dibucaine hydrochloric acid (0.25% to about 1%), dimethisoquin hydrochloric acid (about 0.3% to about 0.5%), dyclonine hydrochloric acid (about 0.5% to about 1%), lidocaine (about 0.5% to about 5%), lidocaine hydrochloric acid (about 0.5% to about 5%), pramoxine hydrochloric acid (about 0.5% to about 1%), tetracaine (about 1% to about 2%), tetracaine hydrochloric acid (about 1% to about 2%), benzyl alcohol (about 10% to about 33%), camphor (about 0.1% to about 3%), juniper tar (about 1% to about 5%), menthol (about 0.1% to about 1%), phenol (about 0.5% to about 1.5%), phenolate sodium (about 0.5% to about 1.5%), resorcinol (about 0.5% to about 3%), diphenhydramine hydrochloric acid (about 1% to about 2%), tripelennamine hydrochloric acid (about 0.5% to about 2%), hydrocortisone (about 0.1% to about 5%, preferably about 0.5% to about 2.5%), and combinations thereof. The compositions of the present invention may optionally also comprise cosmetic anti-itch ingredients such as, for example, Symcalmin® (Symrise GmbH & Co., Holzminden, Germany), which is an oat based anti-itch ingredient, also known as “pentylene glycol and butylene glycol and dihydroavenanthramide D”. Symcalmin® may be present in the composition in an amount of from about 0.1% (by weight of the composition) to about 2% (by weight of the composition).

Optionally, the compositions may additionally comprise sunscreen actives. Suitable sunscreen actives for inclusion in the compositions of the present disclosure include benzophenone-8, butyl methoxydibenzoymethane, cinoxate, DEA-methoxycinnamate, digalloyl trioleate, 1-(3,4-dimethoxyphenyl)-4,4-dimethyl-1,3-pentanediene, ethyl dihydroxypropyl PABA, ethylhexyl dimethyl PABA, ethylhexyl methoxycinnamate, ethylhexyl salicylate, 4-(2-Beta-Blucopyranosiloxy) propoxy-2-hydroxybenzophenone, glyceryl PABA, homosalate, mentyl anthranilate, octocrylene, PABA, phenylbenzimidazole sulfonic acid, red petrolatum, TEA salicylate, titanium dioxide, zinc oxide, surface treated titanium dioxide, surface treated zinc oxide, Spirulina Platensis Powder, Vitis Vinifera seed extract, Helianthus Annus seed extract, tocopherol, terephthalidene dicamphor sulfonic acid, drometrizole trisiloxane, benzylylidene malonate polysiloxane, diethylhexylbutamido triazone, methylene-bis-benzotriazolyl tetramethylbutylphenol, disodium phenyl dibenzimidazole tetrasulfonate, bis-ethylhexyloxyphenol methoxyphenyl triazine, diethylamino hydroxybenzoyl hexyl benzoate, and combinations thereof.

The compositions of the present disclosure may additionally include adjunct components conventionally found in pharmaceutical compositions in their art-established fashion and at their art-established levels. For example, the compositions may comprise additional compatible pharmaceutically active materials for combination therapy, such as antimicrobials, antioxidants, anti-parasitic agents, antipruritics, antifungals, antiseptic actives, biological actives, astringents, keratolytic actives, local anaesthetics, anti-stinging agents, anti-reddening agents, skin soothing agents, and combinations thereof. Other suitable additives that may be included in the compositions of the present disclosure include colorants, deodorants, fragrances, perfumes, emulsifiers, anti-foaming agents, lubricants, natural moisturizing agents, skin conditioning agents, skin protectants and skin benefit agents (e.g., aloe vera and laponite), solvents, solubilizing agents, suspending agents, wetting agents, humectants, preservatives, propellants, dyes and/or pigments, and combinations thereof.

In one embodiment of the present disclosure, the composition, or one or more components of the composition such as the oleocanthal and/or NSAID, may be encapsulated in a shell material prior to being formulated into the composition or introduced into or onto a personal care product or barrier substrate. For instance, the microcapsules may be used to gradually release the oleocanthal and/or NSAID upon an increase in temperature or physical contact, such as when the composition (or personal care product comprising the composition) is contacted with the skin of a user. Suitable microencapsulation shell materials include cellulose-based polymeric materials (e.g., ethyl cellulose), carbohydrate-based materials (e.g., cationic starches and sugars), polyglycolic acid, polylactic acid, and lactic acid-based aliphatic polyesters, and materials derived therefrom (e.g., dextrins and cyclodextrins) as well as other materials compatible with human tissues.

The microencapsulation shell thickness may vary depending upon the composition's formulation, and is generally manufactured to allow the encapsulated composition or component to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should be constructed such that humidity from atmospheric conditions during storage, shipment, or wear will not cause a breakdown of the microencapsulation layer and result in a release of the composition or component.

Microencapsulated compositions or components should be of a size such that the user cannot feel the encapsulated shell on the skin during use. Typically, the capsules have a diameter of no more than about 25 micrometers, and desirably no more than about 10 micrometers. At these sizes, there is no “gritty” or “scratchy” feeling when the composition contacts the skin.

The compositions of the present disclosure may be prepared and applied topically in any suitable form, but preferably are prepared in forms including, without limitation, gels, balms, lotions, suspensions, creams, milks, salves, ointments, sprays, emulsions, oils, resins, foams, solid sticks, aerosols, and the like. Specific examples of products the compositions may be incorporated into include foot creams and wraps, hand creams, topical analgesics, sunburn relief gels and creams, suntan lotions, insect bite relief sprays and/or lotions, diaper rash creams, anti-irritation or anti-inflammatory creams, oily or oily-alcoholic lotions, oily-alcoholic, oily-aqueous, or aqueous-alcoholic gels, ointments, and the like.

As noted above, the compositions may be used to treat inflammation and/or pain, such as is commonly associated with different types of arthritic disorders, injury related discomfort, and other such conditions. The composition may be applied to a body portion, such as a hand, foot, knee, elbow, and the like to treat pain and/or inflammation of the body portion. The composition may be applied by any suitable means, such as rubbing, spraying, rolling, wiping, and the like, and massaged into the body portion to be treated.

In one particular aspect, the compositions are used in combination with a barrier substrate. For instance, the composition may be applied to the body portion to be treated as described above. Optionally, the treated body portion may then be then covered by a barrier substrate. Covering the treated body portion with a barrier substrate helps prevent the composition from rubbing off of the treated body portion onto garments or other objects. Additionally, the presence of a barrier substrate may help maintain a warm environment over the treated body portion, leading to even more effective penetration of the active components (i.e., NSAID and oleocanthal and/or related compounds) through the skin.

Thus, in one aspect, the present disclosure is directed to a method for relieving inflammation and/or pain in a body portion. The method comprises applying a topical composition of the present disclosure to the body portion, and covering the body portion with a barrier substrate. Preferably, the barrier substrate will remain covering the body portion for the duration of treatment. In one aspect, the barrier substrate will remain covering the body portion for from about 1 hour to about 5 hours. Once treatment is complete, the barrier substrate may be removed, and the body portion may be washed to remove any residual composition.

The barrier substrate may be any substrate that may be used to cover a body portion treated with a composition of the present disclosure. Examples of suitable barrier substrates include gloves, socks, wraps, kneepads, elbow pads, headbands, wristbands, patches, thin film (e.g., plastic wrap), and the like.

In one particular embodiment, the barrier substrate is a glove(s) Preferably, the glove will be air permeable, to allow the user's skin to breath, but will be sufficiently impermeable to the composition to prevent the composition from seeping through the glove during wear. Examples of suitable gloves are described in, for example, U.S. Patent App. Publ. No. 2007/0000021, herein incorporated by reference in its entirety.

In one particular embodiment, the composition may be applied to a body portion, and the barrier substrate placed over the treated body portion prior to engaging in activities that may cause pain or inflammation in the body portion. For instance, many people suffer from pain and/or inflammation in the hands as a result of arthritis. Thus, prior to engaging in activities involving use of the hands such as gardening, etc., the composition of the present disclosure may be applied to the hands and a barrier substrate, such as gloves, may be placed over the treated hands. Use of the gloves (or other barrier substrate) in combination with the composition prevents the composition from rubbing off during the activity, while still giving needed pain relief while using the hands.

The composition of the present disclosure may also be impregnated into or onto the barrier substrate. For instance, the barrier substrate will typically comprise an interior surface for contacting the body portion to be treated, and an exterior surface opposite the interior surface. In one embodiment, the composition is impregnated into or onto the interior surface of the barrier substrate. In this embodiment, the body portion is covered with the barrier substrate and the composition is transferred from the interior surface of the barrier substrate to the body portion when the barrier substrate contacts the body portion. Typically, the interior surface of the barrier substrate will comprise from about 5% (by weight of the substrate) to about 1000% (by weight of the substrate)

Thus, in another aspect, the present disclosure is directed to a method for relieving pain and/or inflammation in a body portion. The method comprises covering the body portion with a barrier substrate, the barrier substrate having an interior surface for contacting the body portion, wherein the interior surface of the barrier substrate comprises a topical composition of the present disclosure.

In one preferred embodiment, the barrier substrate is a glove(s). The composition may be impregnated onto the interior surface of the glove, and the glove placed over the hand of a user to relieve pain and/or inflammation.

In another embodiment, the barrier substrate is a transdermal patch, and the compositions are incorporated therein. Methods for preparing transdermal patches are well known in the art. In one particular example, the patch may be a laminated composite, which contains one or more drug reservoir layers (containing the oleocanthal), a backing layer, and optionally one or more additional layers (e.g., additional drug reservoir layers and/or a strippable protective release liner).

The backing layer, which may be adhered to the drug reservoir layer, serves as the upper layer of the patch during use, and functions as the primary structural element of the patch. The backing layer is made of a sheet or film of a preferably flexible elastomeric material that is substantially impermeable to the oleocanthal or oleocanthal composition. The thickness of the layer is not particularly limited and can be appropriately chosen depending on the application, but will typically be on the order of 1.0 to about 4.0 millimeters in thickness. Preferably, the backing layer is composed of a material that permits the patch to follow the contours of the skin, such that it may be worn comfortably on any skin area, e.g., at joints or other points of flexure. In this way, in response to normal mechanical strain, there is little or no likelihood of the patch disengaging from the skin due to differences in the flexibility or resiliency of the skin and the patch. Examples of polymers useful for the backing layer include polyethylene, polypropylene, polyesters, polyurethanes, polyvinyl chloride, polyethylene vinyl acetate, polyvinylidene chloride, block copolymers, nylon, an unwoven fabric, and the like. The backing layer may also comprise laminates of one or more of the foregoing polymers.

The drug reservoir layer typically comprises a contact adhesive which is a pressure-sensitive adhesive suitable for long-term skin contact. The adhesive is preferably also physically and chemically compatible with the NSAID and oleocanthal and with any carriers or vehicles incorporated into an NSAID and oleocanthal composition Further, the adhesive selected for use in the reservoir layer is preferably such that the oleocanthal is at least somewhat soluble in the adhesive. The thickness of the drug reservoir layer is not particularly limited, but will generally be in the range of about 2 to about 4 millimeters in thickness. Suitable adhesives for use in the drug reservoir include polysiloxanes, polyacrylates, polyurethanes, tacky rubbers such as polyisobutylene, and the like. Particularly preferred contact adhesives for use in the drug reservoir herein are cross-linked acrylates.

The adhesive is typically gelled with oleocanthal and NSAID or an NSAID/oleocanthal-containing composition to form the drug reservoir layer. Advantageously, it has been discovered that the transdermal patch, and in particular the drug reservoir layer, may also be constructed using materials that disrupt the stratum corneum, thus facilitating transport of the NSAID and oleocanthal through the stratum corneum. In particular, the drug reservoir layer of the patch may comprise short, stiff, fibers, such as hollow fibers, microfibers, nanofibers, and combinations thereof, gelled in a solution containing NSAID and oleocanthal. Preferably, the fibers are sufficiently sharp that they are able to penetrate, at least partially, the stratum corneum, thus allowing passage of the NSAID and oleocanthal through the stratum corneum.

In one embodiment, the fibers are hollow fibers. Preferably, the hollow fibers have a submicron diameter, but are several microns in length (i.e., have a length sufficient to penetrate at least partially into the stratum corneum). In certain embodiments, the fibers have a diameter of from about 0.1 μm to about 10 μm, more preferably from about 0.2 μm to about 5 μm, and have a length of from about 1 μm to about 100 μm, and more preferably from about 2 μm to about 25 μm. Examples of suitable fibers include, but are not limited to, hollow keratin fibers, carbon nanotubes, wood pulp fiber, processed wood pulp fibers, glass hollow fibers, ceramic fibers (e.g., from aluminum oxides), and fibrous minerals such as sepiolite, attapulgite, and the like. A number of suitable fibers are commercially available, for example, from Engelhard (Iselin, N.J.) and Dupont.

The fibers may be gelled with NSAID and oleocanthal, and optionally suitable carrier materials, additional skin penetration enhancers, polar co-solvents, such as those described above, and/or adhesives to form the drug reservoir layer. Preferably, this composition comprises from about 5% (by weight) to about 50% (by weight) fibers, and more preferably from about 10% (by weight) to about 35% (by weight) fibers. Typically, the composition will comprise oleocanthal in an amount effective to reduce inflammation and/or pain, and preferably at least about 0.04% (by weight of the composition) of oleocanthal, more preferably from about 0.04% (by weight of the composition) to about 10.0% (by weight of the composition) of oleocanthal, more preferably from about 0.10% (by weight of the composition) to about 5.0% (by weight of the composition) of oleocanthal, and still more preferably about 0.50% (by weight of the composition) of oleocanthal. Optionally, the composition may further comprise skin penetration enhancers, polar co-solvents, anti-itch ingredients, and the like, in suitable amounts, such as those described herein. In certain embodiments, the drug reservoir layer may further comprise additional suitable carrier materials, such as those described herein. Optionally, an adhesive, such as those described above, may be included in the composition in any suitable amount, for example, about 10% (by weight) or less,

In one particularly preferred embodiment, the composition comprises from about 10% to about 50% (by weight) hollow fibers, from about 0.04% (by weight of the composition) to about 10.0% (by weight of the composition) of oleocanthal, from about 0.02% (by weight of the composition) to about 5.0% (by weight of the composition) of an NSAID, and from about 0.01% (by weight of the composition) to about 25% (by weight of the composition) of a skin penetration enhancer.

Preferably, the fibers, the NSAID, oleocanthal, and optionally the adhesive, skin penetration enhancers, polar co-solvents, and/or carrier materials are gelled together to form the drug reservoir layer. For example, these components may be combined with oil soluble polymeric substances suitable for forming a gel. Preferably, the oil soluble polymeric materials included in the drug reservoir layer are present in an amount from about 0.5% to about 50% by weight of the composition and more preferably from about 5% to about 25% by weight of the composition.

Optionally, the patch may further comprise a release liner. The release liner is a disposable element which serves to protect the patch prior to application. Typically, the release liner is formed from a material impermeable to the NSAID, oleocanthal, any carriers or vehicles, and adhesive, and is easily stripped from the contact adhesive that serves as part of the drug reservoir layer. Preferred release liners for use herein are those which are generally suitable for use in conjunction with pressure-sensitive adhesives, such as silanized polyester films, among others.

In general, the patches are fabricated using methods standard in the art. For example, the patch may be prepared by spreading and coating the NSAID/oleocanthal/adhesive composition onto an appropriate substrate (e.g., the backing layer or the release liner) to form a drug reservoir layer. Other layers may then be laminated to this initial structure.

The patch may be applied directly to the skin of a user. Advantageously, the penetration of the NSAID and oleocanthal into the skin and through the stratum corneum is enhanced by the presence of the hollow fibers in the drug reservoir layer. In certain instances, the hollow interior of the fibers may act as a reservoir for the NSAID and oleocanthal, thus allowing slow, continuous release of the NSAID and oleocanthal to the user.

In one particular aspect, the compositions of the present disclosure may be used in combination with a product, such as a personal care product. More particularly, the composition may be incorporated into or onto a substrate, such as a wipe substrate, an absorbent substrate, a fabric or cloth substrate, or a tissue substrate, among others. In this instance, the substrate of the personal care product may be used to transfer the composition to the skin of the user at the point where the product comes in contact with the skin. In certain embodiments, the personal care product substrate may also act as a barrier substrate, as described above. Numerous products can be used in combination with the NSAID and oleocanthal-containing compositions described herein in accordance with the present disclosure to reduce inflammation and/or pain in a user. For example, the NSAID and oleocanthal-containing compositions may be incorporated into personal care products, such as wipes, absorbent articles, bath tissues, cloths, and patches among others. More particularly, the NSAID and oleocanthal may be incorporated into wipes such as wet wipes, dry wipes, hand wipes, face wipes, cosmetic wipes, and the like, absorbent articles (in particular medical wraps and bandages).

Although discussed primarily in combination with a wipe substrate, it should be understood that the NSAID and oleocanthal-containing compositions can also be used in combination with other numerous personal care products, such as those described above. Materials suitable for use as the substrate of the wipe are well known to those skilled in the art, and typically include a fibrous sheet material, which may be either woven or nonwoven. For example, the wipes incorporating the oleocanthal described herein to reduce inflammation and/or pain may include nonwoven fibrous sheet materials, which include meltblown, coform, air-laid, bonded-carded web materials, hydroentangled materials, and combinations thereof. Such materials can be comprised of synthetic or natural fibers, or a combination thereof. Typically, wipes define a basis weight of from about 25 to about 120 grams per square meter and desirably from about 40 to about 90 grams per square meter.

In a particular embodiment, the wipes incorporating the compositions described herein comprise a coform basesheet of polymeric microfibers and cellulosic fibers having a basis weight of from about 60 to about 80 grams per square meter and desirably about 75 grams per square meter. Such coform basesheets are manufactured generally as described in U.S. Pat. No. 4,100,324, which is incorporated by reference. Typically, such coform basesheets comprise a gas-formed matrix of thermoplastic polymeric meltblown microfibers, such as, for example, polypropylene microfibers, and cellulosic fibers, such as, for example, wood pulp fibers.

The relative percentages of the polymeric microfibers and cellulosic fibers in the coform basesheet can vary over a wide range depending upon the desired characteristics of the wipes. For example, the coform basesheet may comprise from about 20 to about 100 weight percent, desirably from about 20 to about 60 weight percent, and more desirably from about 30 to about 40 weight percent of the polymeric microfibers based on the dry weight of the coform basesheet being used to provide the wipes.

Alternatively, the wipes incorporating the compositions described herein can comprise a composite, which includes multiple layers of materials such as those described in U.S. Pat. No. 6,028,018, which is incorporated by reference. For example, the wipes may include a three layer composite, which includes an elastomeric film or meltblown layer between two coform layers as described above. In such a configuration, the coform layers may define a basis weight of from about 15 to about 30 grams per square meter and the elastomeric layer may include a film material such as a polyethylene metallocene film.

As mentioned above, one type of wipe suitable for use in combination with the compositions described herein to reduce inflammation and/or pain include wet wipes, which, in addition to the wipe substrate, comprise a liquid solution or formulation. The liquid solution or formulation can be any liquid, which can be absorbed into the wet wipe basesheet and may include any suitable components, which provide the desired wiping properties. For example, the components may include water, emollients, surfactants, fragrances, preservatives, chelating agents, pH buffers, or combinations thereof as are well known to those skilled in the art. Further, the liquid may also contain medicaments and/or antimicrobials.

The amount of liquid contained within each wet wipe may vary depending upon the type of material being used to provide the wet wipe, the type of liquid being used, the type of container being used to store the wet wipes, and the desired end use of the wet wipe. Generally, each wet wipe can contain from about 150 to about 600 weight percent and desirably from about 250 to about 450 weight percent liquid based on the dry weight of the wipe for improved wiping. In a particular aspect, the amount of liquid contained within the wet wipe is from about 300 to about 400 weight percent and desirably about 330 weight percent based on the dry weight of the wet wipe. If the amount of liquid is less than the above-identified ranges, the wet wipe may be too dry and may not adequately perform. If the amount of liquid is greater than the above-identified ranges, the wet wipe may be oversaturated and soggy and the liquid may pool in the bottom of the container.

Each wet wipe is generally rectangular in shape and may have any suitable unfolded width and length. For example, the wet wipe may have an unfolded length of from about 2.0 to about 80.0 centimeters and desirably from about 10.0 to about 25.0 centimeters and an unfolded width of from about 2.0 to about 80.0 centimeters and desirably from about 10.0 to about 25.0 centimeters. Typically, each individual wet wipe is arranged in a folded configuration and stacked one on top of the other to provide a stack of wet wipes. Such folded configurations are well known to those skilled in the art and include c-folded, z-folded, quarter-folded configurations and the like. The stack of folded wet wipes may be placed in the interior of a container, such as a plastic tub, to provide a package of wet wipes for eventual sale to the consumer. Alternatively, the wet wipes may include a continuous strip of material which has perforations between each wipe and which may be arranged in a stack or wound into a roll for dispensing.

In one embodiment of the present disclosure, the NSAID and oleocanthal-containing compositions are introduced into or onto a fibrous wipe substrate, absorbent substrate, a fabric or cloth substrate, or a tissue substrate. When the substrate contacts the skin of the user, the composition contacts the skin, and may be actually transferred to the skin, whereupon it may be absorbed into skin to reduce inflammation and/or pain in the user. In a specific embodiment, the compound is introduced into the liquid formulation of a wet wipe.

The NSAID and oleocanthal compositions described herein may be applied to the substrate using any conventional methods, such as dipping, spraying, printing, coating, extrusion, ink jet printing, and combinations thereof. Preferably, the composition is introduced onto the substrate in an amount of from about 5% by weight of the substrate to about 1000% by weight of the substrate, desirably from about 100% by weight of the substrate to about 500% by weight of the substrate, and more desirably from about 150% by weight of the substrate to about 300% by weight of the substrate. As noted above, the wipe may be used to apply the composition to the body portion to be treated.

In another aspect, the present disclosure is directed to a system for relieving pain and/or inflammation in a body portion. The system may comprise a barrier substrate comprising an interior surface for contacting the body portion and an exterior surface opposite the interior surface, and a topical composition of the present disclosure. Optionally, the system may further comprise a wipe or other personal care product, such as described herein, that may be used to apply the composition to the body portion. In one aspect, the system components may be packaged together in a common package, such as a kit. As noted above, the composition may be impregnated into or onto the barrier substrate and/or personal care product, or may be packaged with the barrier substrate and/or personal care product in a separate container.

Having described the disclosure in detail, it will be apparent that modifications and variations are possible without departing from the scope of the disclosure defined in the appended claims.

### EXAMPLES

The following non-limiting examples are provided to further illustrate the present disclosure.

### Example 1

In this example, an ibuprofen-containing olive oil formulation was produced. The following components were used to prepare the formulation.

TABLE 1

| | Amount (% |
|---|---|
| Component | weight) |
| Ibuprofen | 5.0 |
| Olive oil | 55.0 |
| Beeswax | 2.5 |
| Stearyl alcohol | 2.5 |
| Menthol | 0.8 |
| Eumulgin SG | 1.5 |
| Dicapryl adipate | 29.2 |
| Silica | 3.0 |

The formulation was prepared by mixing together the olive oil, ibuprofen, beeswax, stearyl alcohol, and dicapryl adipate and heating to 70-75° C. to allow the solids to melt. Heating was discontinued and the silica and Eumulgin SG were added to the mixture. The mixture was allowed to cool to ambient temperature, at which point the menthol was added.

### Example 2

In this example, an oleocanthal and ibuprofen-containing olive oil formulation is produced. The following components are used to prepare the formulation.

TABLE 2

| | Amount (% |
|---|---|
| Component | weight) |
| Ibuprofen | 5.0 |
| Olive oil | 50.0 |
| Petrolatum | 10.0 |
| Capric caprylic | 18.8 |
| triglyceride | |
| Beeswax | 1.0 |
| Propylene glycol | 5.0 |
| Stearyl alcohol | 3.0 |
| Glyceryl stearate | 1.0 |
| Menthol | 1.0 |
| Lauric acid | 5.0 |
| Vitamin E | 0.2 |

### Example 3

In this example, an oleocanthal and ibuprofen-containing olive oil formulation is produced. The following components are used to prepare the formulation.

TABLE 3

| | Amount (% |
|---|---|
| Component | weight) |
| Ibuprofen | 5.0 |
| Olive oil | 50.0 |
| Petrolatum | 5.0 |
| Capric caprylic | 10.0 |
| triglyceride | |
| Beeswax | 1.0 |
| Propylene glycol | 10.0 |
| Stearyl alcohol | 4.0 |
| Ethylheptamethyltrisiloxane | 8.8 |
| Menthol | 1.0 |
| Lauric acid | 5.0 |
| Vitamin E | 0.2 |

### Example 4

In this example, an oleocanthal and ibuprofen-containing olive oil formulation is produced. The following components are used to prepare the formulation.

TABLE 4

| | Amount (% |
|---|---|
| Component | weight) |
| Ibuprofen | 5.0 |
| Olive oil | 50.0 |
| Petrolatum | 5.0 |
| Capric caprylic | 9.8 |
| triglyceride | |
| Beeswax | 2.0 |
| Propylene glycol | 5.0 |
| Xylitol | 2.0 |
| Stearyl alcohol | 3.0 |
| Cetyl dimethicone | 5.0 |
| (Abil 9801) | |
| Hydrogenated | 5.0 |
| Polydecene | |
| Glyceryl stearate | 2.0 |
| Menthone (peppermint) | 1.0 |
| Lauric acid | 5.0 |
| Vitamin E | 0.2 |

### Example 5

In this example, an oleocanthal and ibuprofen-containing olive oil formulation is produced. The following components are used to prepare the formulation.

TABLE 5

| | Amount (% |
|---|---|
| Component | weight) |
| Ibuprofen | 5.0 |
| Olive oil | 50.0 |
| Capric caprylic | 7.8 |
| triglyceride | |
| Beeswax | 1.0 |
| Mineral oil | 10.0 |
| Stearyl alcohol | 5.0 |
| Isodecyl neopentanoate | 8.0 |
| Dicapryl adipate | 7.0 |
| Menthone | 1.0 |
| Lauric acid | 5.0 |
| Butylated hydroxytoluene | 0.2 |

### Example 6

In this example, an oleocanthal and ibuprofen-containing olive oil formulation is produced. The following components are used to prepare the formulation.

TABLE 6

| | Amount (% |
|---|---|
| Component | weight) |
| Ibuprofen | 5.0 |
| Olive oil | 50.0 |
| Petrolatum | 8.0 |
| Capric caprylic | 8.0 |
| triglyceride | |
| Beeswax | 1.5 |
| Propylene glycol | 3.3 |
| Stearyl alcohol | 3.0 |
| Decyl oleate | 7.0 |
| Dicapryl maleate | 7.0 |
| Glyceryl stearate | 1.0 |
| 1,8-cineole | 1.0 |
| (Eucaliptol) | |
| Lauric acid | 5.0 |
| Butylated | 0.2 |
| hydroxytoluene | |

### Example 7

In this example, an oleocanthal and ibuprofen-containing olive oil formulation is produced. The following components are used to prepare the formulation.

TABLE 7

| | Amount (% |
|---|---|
| Component | weight) |
| Ibuprofen | 5.0 |
| Olive oil | 50.0 |
| Petrolatum | 5.0 |
| Capric caprylic | 10.0 |
| triglyceride | |
| Propylene glycol | 4.8 |
| Stearyl alcohol | 5.0 |
| Ethylhexyl palmitate | 7.0 |
| Isodecane | 7.0 |
| 1,8-cineole | 1.0 |
| (Eucalyptol) | |
| Lauric acid | 5.0 |
| Butylated | 0.2 |
| hydroxytoluene | |

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above compositions, products, and methods without departing from the scope of the disclosure, it is intended that all matter contained in the above description shall be interpreted as illustrative and not in a limiting sense.

What is claimed is:

1. A composition for relief of inflammation or pain, the composition comprising a pharmaceutically acceptable carrier, at least about 0.04% (by weight of the composition) of a first active ingredient, and an effective amount of a second active ingredient, wherein the first active ingredient has the general structure: 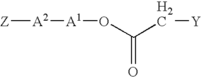
wherein Z is 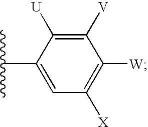
u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH((OCH₃); A¹ is selected from the group consisting of CH₂, CHCH₃, CH(CH₂CH₃), and C(CH₃)₂, CH(NH₂); and Y is selected from the group consisting of 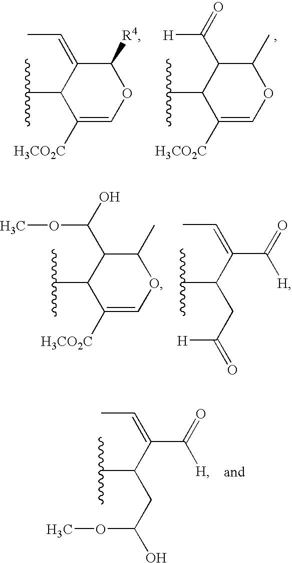 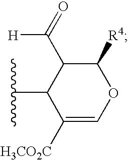
wherein R⁴ is selected from the group consisting of —H and 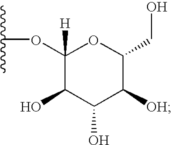
wherein the second active ingredient is a non-steroidal anti-inflammatory drug.

2. The composition of claim 1 wherein the first active ingredient is selected from the group consisting

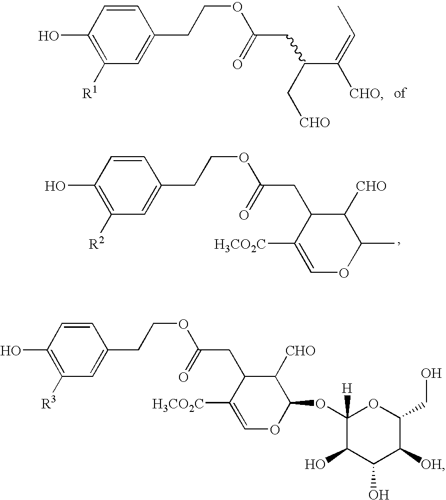

3. The composition of claim 2 wherein the first active ingredient is oleocanthal.

4. The composition of claim 1 wherein the composition comprises from about 0.04% (by weight of the composition) to about 10.0% (by weight of the composition) of the first active ingredient.

5. The composition of claim 1 wherein the second active ingredient is selected from the group consisting of ibuprofen, aspirin, difunisal, etodolac, indometacin, nabumeton, sulindac, tolmetin, caprofen, fenbufen, flubiprofen, ketoprofen, ketorolac, loxoprofen, naproxen, oxaprozin, tiaprofenic acid, suprofen, mefenaminic acid, phenylbutazone, piroxicam, meloxicam, celecoxib, etoricoxib, lumiracoxib, parecoxib, rofecoxib, valdecoxib, nimesulide, and combinations thereof.

6. The composition of claim 1 wherein the composition comprises from about 0.02% (by weight of the composition) to about 10.0% (by weight of the composition) of the second active ingredient.

7. The composition of claim 1 wherein the composition comprises an aqueous phase, wherein at least about 50% of the second active ingredient present in the aqueous phase of the composition is in non-ionized form.

8. The composition of claim 1 wherein the carrier is olive oil.

9. The composition of claim 1 further comprising a skin penetration enhancer.

10. The composition of claim 9 wherein the composition comprises from about 0.01% (by weight of the composition) to about 25% (by weight of the composition) of the skin penetration enhancer.

11. The composition of claim 1 wherein the composition comprises less than about 10% (by weight of the composition) of water.

12. The composition of claim 1 wherein the composition further comprises a polar co-solvent.

13. A composition for relief of pain or inflammation, the composition comprising a pharmaceutically acceptable carrier, a first active ingredient, a second active ingredient, and a skin penetration enhancer; wherein the second active ingredient is a non-steroidal anti-inflammatory drug, and the first active ingredient has the general structure: 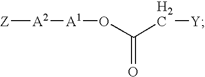
wherein Z is 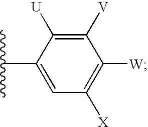
u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH(OCH₃); A¹ is selected from the group consisting of CH₂, CHCH₃, CH(CH₂CH₃), and C(CH₃) ₂, CH(NH₂); and Y is selected from the group consisting of 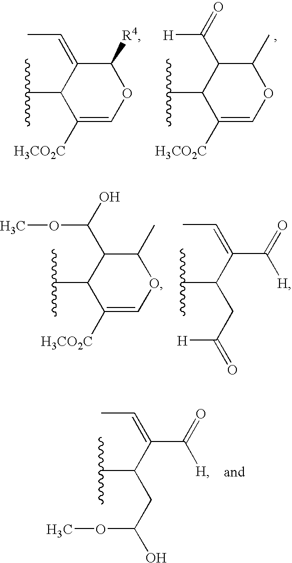 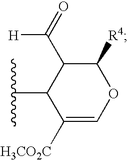
wherein R⁴ is selected from the group consisting of —H and

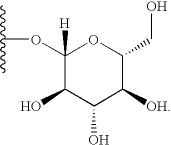

14. A method for relieving pain or inflammation in a body portion, the method comprising:
applying a composition to the body portion, the composition comprising a pharmaceutically acceptable carrier, at least about 0.04% (by weight of the composition) of a first active ingredient, and a second active ingredient; and
covering the body portion with a barrier substrate;
wherein the second active ingredient is a non-steroidal anti-inflammatory drug and the first active ingredient has the general structure; 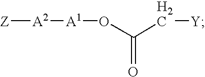
wherein Z is 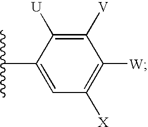
u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH(OCH₃); A¹ is selected from the group consisting of CH₂, CHCH₃, CH(CH₂CH₃), and C(CH₃)₂, CH(NH₂); and Y is selected from the group consisting of 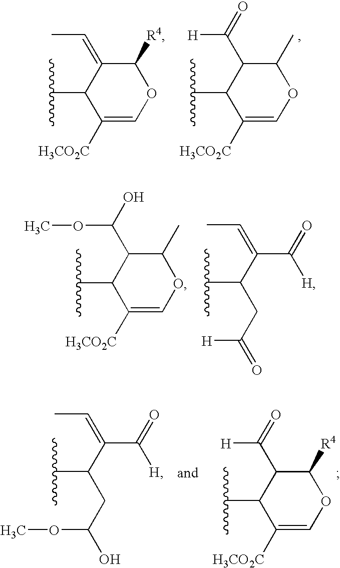
wherein R⁴ is selected form the group consisting of —H and

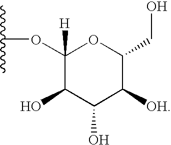

15. The method of claim 14 wherein the barrier substrate is selected from the group consisting of personal care products, gloves, socks, wraps, knee pads, elbow pads, headbands, wristbands, patches, and thin films.

16. The method of claim 15 wherein the barrier substrate is a glove.

17. A method for relieving pain or inflammation in a body portion, the method comprising:
covering the body portion with a barrier substrate, the barrier substrate having an interior surface for contacting the body portion;
wherein the interior surface of the barrier substrate comprises a composition, the composition comprising a pharmaceutically acceptable carrier, at least about 0.04% (by weight of the composition) of a first active ingredient, and a second active ingredient, wherein the second active ingredient is a non-steroidal anti-inflammatory drug, and the first active ingredient has the general structure: 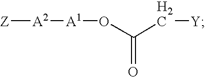
wherein Z is 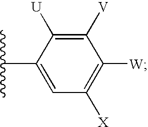
u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH(OCH₃); A¹ is selected from the group consisting of CH₂, CHCH₃, CH(CH₂CH₃), and C(CH₃)₂, CH(NH₂); and Y is selected from the group consisting of 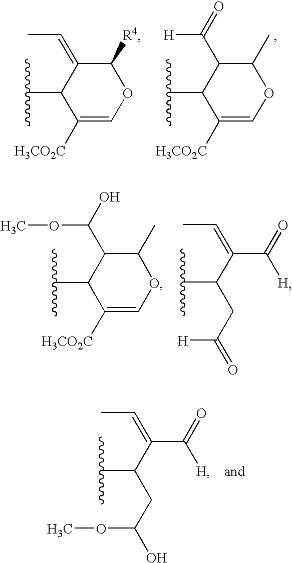 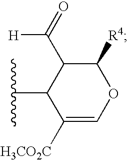
wherein R⁴ is selected form the group consisting of —H and

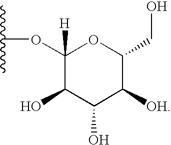

18. The barrier substrate of claim 17 wherein the interior surface comprises from about 5% (by weight of the substrate) to about 1000% (by weight of the substrate) of the composition.

19. The method of claim 17 wherein the barrier substrate is selected from the group consisting of personal care products, gloves, socks, wraps, knee pads, elbow pads, headbands, wristbands, patches, and thin films.

20. The method of claim 19 wherein the barrier substrate is a glove.

21. A system for relieving pain or inflammation in a body portion, the system comprising:
a barrier substrate comprising an interior surface for contacting the body portion and an exterior surface opposite the interior surface; and
a composition comprising a pharmaceutically acceptable carrier, at least about 0.04% (by weight of the composition) of a first active ingredient, and a second active ingredient, wherein the second active ingredient is a non-steroidal anti-inflammatory drug, and the first active ingredient has a general structure: 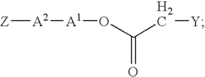
wherein Z is 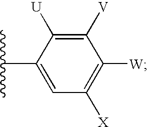
u, v, w, and x are independently selected from the group consisting of —H, —OH, —CH₃, —NH₂, —NHCH₃, —NO₂, —F, —Cl, —Br, —I, —OCH₃, and —CF₃; A² is selected from the group consisting of CH₂, CHCH₃, CH(CH₂CH₃), C(CH₃)₂, CH(OH), and CH(OCH₃); A¹ is selected from the group consisting of CH₂, CHCH₃, CH(CH₂CH₃), and C(CH₃)₂, CH(NH₂); and Y is selected from the group consisting of 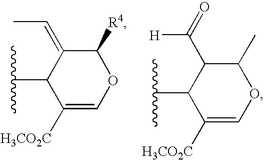 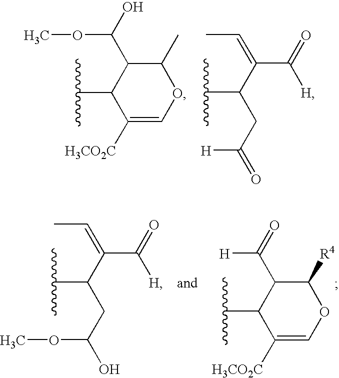
wherein R⁴ is selected form the group consisting of —H and

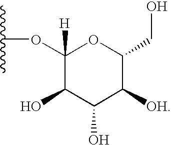

22. The system of claim 21 wherein the barrier substrate and the composition are packaged together in a common package.

23. The system of claim 22 wherein the barrier substrate is selected from the group consisting of personal care products, gloves, socks, wraps, knee pads, elbow pads, headbands, wristbands, patches, and thin films.

24. The system of claim 21 wherein the composition is located on the interior surface of the barrier substrate.
